(19) United States
(12) Patent Application Publication
(10) Pub. No.: US 2005/0049629 A1
(43) Pub. Date: Mar. 3, 2005

(54) Medical clamp

(76) Inventor: Bon Hee Koo, Busanjin-Gu (KR)

Correspondence Address: JORDAN AND HAMBURG LLP, 122 EAST 42ND STREET, SUITE 4000, NEW YORK, NY, 10168, US

(21) Appl. No.: 10/926,526

(22) Filed: Aug. 25, 2004

(30) Foreign Application Priority Data
Aug. 29, 2003 (KR) 20-2003-0027847

(51) Int. Cl. A61B 17/58

(52) U.S. Cl. 606/201

(57) ABSTRACT

Disclosed is a medical clamp for safely and correctly reducing a fractured bone without any wound on the skin of the opposite side of the fractured region when the fractured bone is reduced. The medical clamp includes: two clamp members having handle loops formed on a side thereof and forceps portions sharply formed on the opposite side thereof; and a pin for rotatably coupling the two clamp members with each other. The medical clamp further includes: a coupling ball formed on the tip of one of the forceps portions; a pad having a curved contact surface to be in close contact with a patient's skin, a coupling hole and a slide slot vertically formed in the opposite side of the contact surface and communicating with each other, the coupling hole and the slide slot respectively corresponding to the forceps portion and the coupling ball, and a tapered rotation hole formed in the center of the slide slot.

### BACKGROUND OF THE INVENTION

1. Field of the Invention

The present invention relates to a medical clamp, which is one of surgical instruments and used for reducing a fractured bone of a patient who suffers a fracture of an ankle, and more particularly, to a medical clamp for safely and correctly reducing a fractured bone without any wound on the skin of the opposite side of the fractured region when the fractured bone is reduced.

2. Background of the Related Art

In general, the anklebone region projecting from both sides of a human's ankle, namely medial malleolus region, can be easily fractured in an aspect of the bone structure. In a surgical operation for reducing the fractured anklebone, the fractured anklebone is located on the original position, fastened with a clamp in order not to be moved, and then, fixed with a supplement such as an implant.

As shown in FIG. 4, the medical clamp 1 used for the surgical operation includes two clamp members 2 and 2′ having handle loops 3 formed on a side thereof and forceps portions 4 sharply formed on the opposite side thereof, and a pin 5 for rotatably coupling the two clamp members 2 and 2′ with each other. Such medical clamp 1 can reduce the fractured bone located between the two forceps portions 4. However, the conventional medical clamp 1 has several problems in the bone-setting operation of the anklebone.

A wide pad in contact with the skin must be fixed onto one of the forceps portions 4 with an adhesive tape in order to prevent injury of the non-fractured skin. However, it is difficult to closely contact the pad fixed on the forceps portion 4 with the skin because patients have various body figures.

Furthermore, even though the pad and the forceps portions 4 are in closely adhered state, it is very difficult to reduce the fractured bone by pressing the two clamp members 2 and 2′.

### SUMMARY OF THE INVENTION

Accordingly, the present invention is directed to a medical clamp that substantially obviates one or more problems due to limitations and disadvantages of the related art.

An object of the present invention is to provide a medical clamp, which has a pad capable of being closely fixed onto the skin of the non-fractured bone region is detachably and rotatably coupled with one of forceps portions of clamp members when a fractured bone of the anklebone region is surgically operated, thereby guaranteeing a safe and rapid surgical operation by closely contacting the pad with the skin without any inconvenience to additionally fix the pad to the skin to make a close contact state of the pad with the skin.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is an exploded perspective view of a preferred embodiment of the present invention;

FIG. 2 is a plan sectional view of essential parts of the present invention;

FIG. 3 is a perspective view showing a used state of the present invention; and

FIG. 4 is a perspective view showing a used state of a conventional medical clamp.

### DETAILED DESCRIPTION OF THE PREFERRED EMBODIMENT

Reference will now be made in detail to the preferred embodiments of the present invention, examples of which are illustrated in the accompanying drawings.

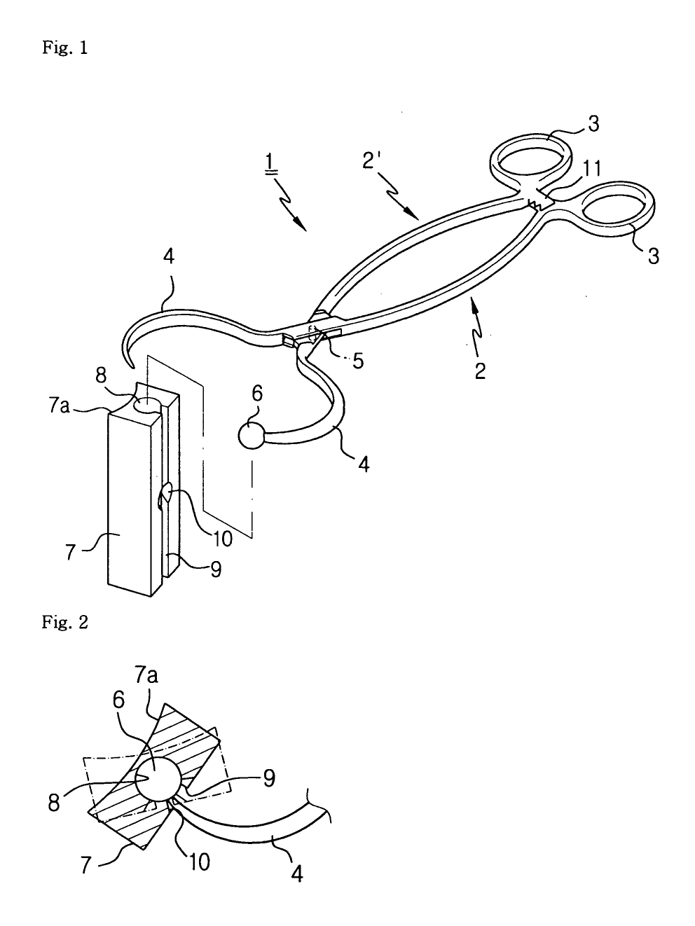

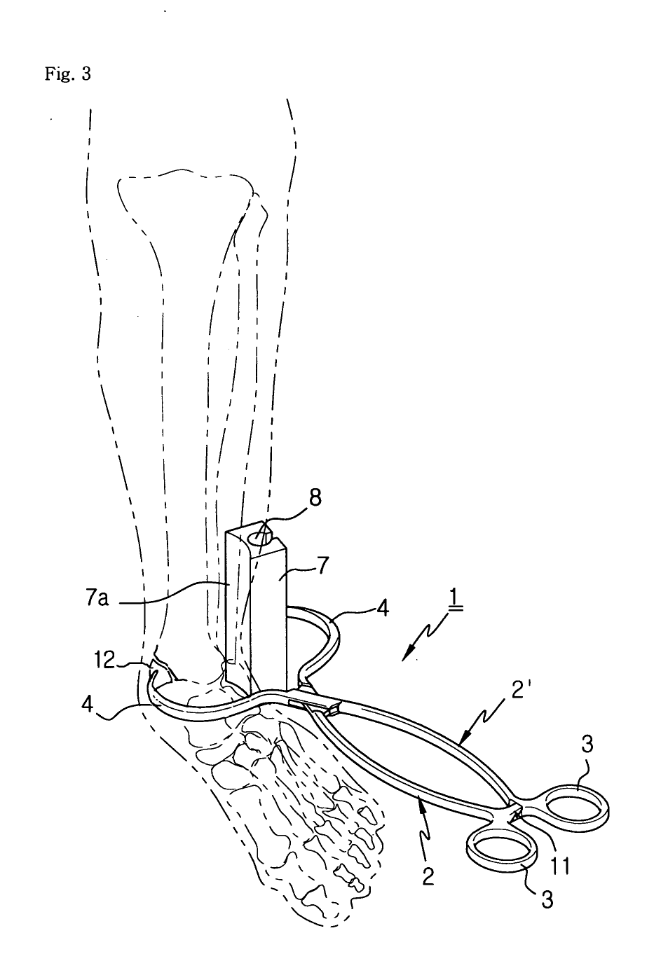

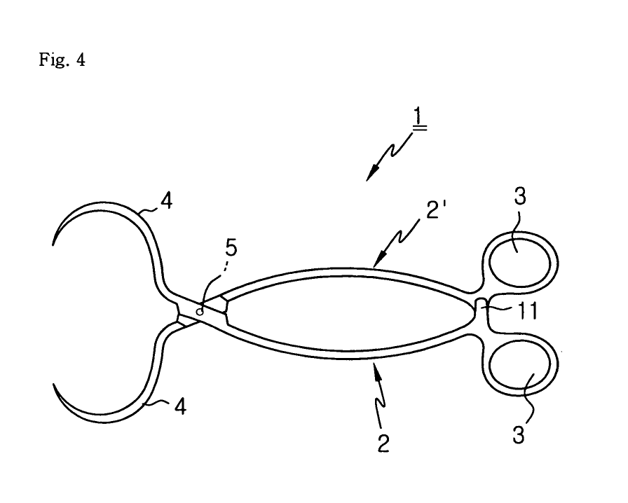

FIG. 1 is an exploded perspective view of a preferred embodiment of the present invention, FIG. 2 is a plan sectional view of essential parts of the present invention, and FIG. 3 is a perspective view showing a used state of the present invention. In the drawings, parts equal or similar to those of the prior art has the same reference numerals as those of the prior art.

The present invention is more improved than the conventional medical clamp 1, which includes two clamp members 2 and 2′ having handle loops 3 formed on a side thereof and forceps portions 4 sharply formed on the opposite side thereof, and a pin 5 for rotatably coupling the two clamp members 2 and 2′ with each other in order to reduce the fractured bone located between the two forceps portions 4.

As shown in the drawings, a medical clamp 1 according to the present invention has a coupling ball 6 formed on the tip of one of the forceps portions 4, and a pad 7 slidably coupled with the tip of one of forceps portion 4.

The pad 7 includes: a contact surface 7a to be in close contact with a patient's skin; a coupling hole 8 and a slide slot 9 vertically formed in the opposite side of the contact surface 7a and communicating with each other, the coupling hole 8 and the slide slot 9 respectively corresponding to the forceps portion 4 and the coupling ball 6; and a tapered rotation hole 10 formed in the center of the slide slot 9 in such a way as to rotate the pad 7 contacting the skin at a predetermined angle in a state where the coupling ball 6 formed on the tip of the forceps portion 4 is coupled with the coupling hole 8 and the slide slot 9. So, the medical clamp 1 according to the present invention can provide a stable and safe reduction of the fractured bone.

Alternatively, the pad 7 may be rotatably fixed to the coupling ball 6 (not shown).

In the drawings, the unexplained reference numeral 11 designates fixing means formed in such a way to be faced with each other in the inward direction of handle loops and having several serrations, and the reference numeral 12 designates fractured bone of the anklebones.

As shown in FIG. 3, the medical clamp 1 according to the present invention is used for the surgical operation of the fractured anklebone region in a state where the pad 7 is coupled with the coupling ball 6.

According to the present invention, the contact surface 7a of the pad 7 slidably coupled with the coupling ball 6 is curved, and the pad 7 can be rotated in a state where the pad 7 is coupled with the coupling ball 6 formed on the forceps portion 4 so as to achieve a stable reduction of the fractured bone region by increasing contact force on the skin.

That is, since the pad 7 has the tapered rotation hole 10 formed in the center of the slide slot 9, the position to perform reduction of the fractured bone is settled in a state where the forceps portion 4 is inserted into the tapered rotation hole 10 when the coupling ball 6 and the forceps portion 4 are coupled with the coupling hole 8 and the slide slot 9, and then, the handle loops 3 formed on the two clamp members 2 and 2′ are pressured. As shown in FIG. 2, the pad 7 is in natural contact with the skin as being freely rotated, so that the present invention can solve the problem of a bad reduction due to deterioration of fixing force of the pad 7. At this time, the tip of the other forceps portion 4 presses the bone 12 to be reduced.

As described above, in a state where the fractured bone region is reduced, as shown in FIG. 3, the two clamp members 2 and 2′ are fixed with the fixing means 11. And, at this time, a supplement such as an implant is fixed.

Meanwhile, in this embodiment, the present invention is used for reduction of the fractured bone of the anklebone region, but it is appreciated that the medical clamp 1 according to the present invention can be used for reducing other fractured bones, such as arms, legs or others.

As described above, the medical clamp 1 according to the present invention is mainly used for reduction of the fractured anklebone, and includes the coupling ball 6 formed on one of the forceps portions 4 and the pad 7 detachably and rotatably coupled with the coupling ball 6 in a slidable way. According to the present invention, the medical clamp 1, which has a simple structure, can be manufactured in a low price. Moreover, since the present invention can implant the supplement in a state where the fractured region to be surgically operated is stably and rapidly reduced, the medical clamp is a medical instrument capable of guaranteeing safety without medical accidents.

The forgoing embodiment is merely exemplary and is not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

1. A medical clamp, which includes two clamp members having handle loops formed on a side thereof and forceps portions sharply formed on the opposite side thereof, and a pin for rotatably coupling the two clamp members with each other, the medical clamp comprising:
a coupling ball formed on the tip of one of the forceps portions;
a pad having a curved contact surface to be in close contact with a patient's skin, a coupling hole and a slide slot vertically formed in the opposite side of the contact surface and communicating with each other, the coupling hole and the slide slot respectively corresponding to the forceps portion and the coupling ball, and a tapered rotation hole formed in the center of the slide slot.
